(19) United States
(12) Patent Application Publication
(10) Pub. No.: US 2007/0141014 A1
(43) Pub. Date: Jun. 21, 2007

(54) Formulation assistants

(76) Inventors: Frank Pflucker, Darmstadt (DE); Hans-Werner Grunewald, Reinheim (DE); Hansjurgen Driller, Gross-Umstadt (DE); Michael Schwarz, Weiterstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/588,461

(22) PCT Filed: Jan. 13, 2005
(86) PCT No.: PCT/EP05/00274

§ 371 Date: Aug. 4, 2006

(30) Foreign Application Priority Data
Feb. 7, 2004 (DE) 10 2004 006 144.0

(51) Int. Cl. A61K 8/42

(52) U.S. Cl. 424/70.31

(57) ABSTRACT

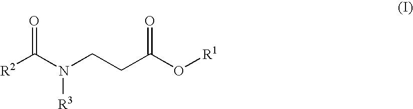

The invention relates to the use of compounds of the formula (I), where R¹, R² and R³ may be identical or different and are selected from: straight-chain or branched C₁- to C₂₄-alkyl groups, straight-chain or branched C₃- to C₂₄-alkenyl groups, straight-chain or branched C₁- to C₂₄-hydroxyalkyl groups, where the hydroxyl group may be bonded to a primary or secondary carbon atom of the chain and furthermore the alkyl chain may also be interrupted by oxygen, and/or C₃- to C₁₀-cycloalkyl groups and/or C₃- to C₁₂-cycloalkenyl groups, where the rings may in each case also be bridged by (CH₂)ₙ groups, where n=1 to 3, as formulation assistants for the preparation of cosmetic or dermatological compositions, to corresponding novel compounds, and to the preparation thereof.

The present invention relates to the use of certain substances as formulation assistants for the preparation of cosmetic or dermatological compositions, to corresponding novel compounds, and to the preparation thereof.

An example of an area of application of the formulation assistants according to the invention is cosmetics. The object of care cosmetics is wherever possible to obtain the impression of youthful skin. In principle, there are various ways of achieving this object. For example, existing skin damage, such as irregular pigmentation or the formation of wrinkles, can be compensated for by covering powders or creams. Another approach is to protect the skin against environmental influences which lead to permanent damage and thus ageing of the skin. The idea is therefore to intervene in a preventative manner and thus to delay the ageing process. One example of this are the UV filters already mentioned, which, as a result of absorption of certain wavelength ranges, prevent or at least reduce skin damage. Whereas in the case of UV filters the damaging event, the UV radiation, is screened off by the skin, another route involves attempting to support the skin's natural defence or repair mechanisms against the damaging event. Finally, a further approach involves compensating for the weakening defence functions of the skin against harmful influences with increasing age by externally supplying substances which are able to replace this diminishing defence or repair function. For example, the skin has the ability to scavenge free radicals formed by external or internal stress factors. This ability diminishes with increasing age, causing the ageing process to accelerate with increasing age.

Skin damage is not caused just by sunlight, but also by other external influences, such as cold or heat. Furthermore, the skin undergoes natural ageing, with the formation of wrinkles and a reduction in the elasticity of the skin.

An extremely wide variety of active ingredients, for example classical active ingredients, such as UV filters and antioxidants, or modem active ingredients which engage directly in the biochemical processes in the skin, are known which are suitable for contributing to the prophylaxis, treatment or amelioration of the symptoms of the skin-stress phenomena described above.

However, a further difficulty in the preparation of cosmetics consists in that many of these active ingredients which are to be incorporated into cosmetic compositions are often insoluble or only sparingly soluble, are unstable and can be damaged in the composition or have to be employed in large amounts or reduce the stability of the resultant formulations.

The damage can be caused, for example, by reaction with atmospheric oxygen or by absorption of UV rays. Due to their structural change, the molecules damaged in this way can, for example, change their colour and/or lose their efficacy.

There is therefore a demand for assistants which are suitable, in combination with the respective active ingredients, for solving one or more of the said problems.

The object of the invention is therefore to provide a formulation assistant which can be incorporated into cosmetic compositions and, in combination with suitable active ingredients, simplifies incorporation thereof and/or improves the efficacy and/or stability thereof and/or increases the stability of the resultant formulation.

Surprisingly, this object is achieved by the use of certain β-alanine derivatives as formulation assistants for the preparation of cosmetic or dermatological compositions.

The present invention therefore relates firstly to the use of a compound of the formula I 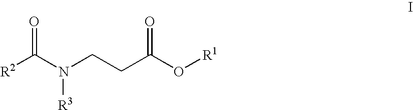
where R¹, R² and R³ may be identical or different and are selected from
straight-chain or branched C₁- to C₂₄-alkyl groups,
straight-chain or branched C₃- to C₂₄-alkenyl groups,
straight-chain or branched C₁- to C₂₄-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
   as formulation assistant in the preparation of cosmetic or dermatological compositions.

In preferred embodiments of the present invention, the said compounds can thus serve as solubilisers, penetration enhancers and/or action enhancers.

Some of the compounds of the formula I having radicals as defined above or structurally similar compounds are known from the literature:
Ethyl 3-(acetylbutylamino)propionate is a known insect repellent which is marketed by Merck under the trade name IR3535®.
The suitability of further β-alanine derivatives as insect repellents is investigated in M. Klier, F. Kuhlow, J. Soc. Cos. Chem., 27, pp. 141-153 (1976).

It may be preferred in accordance with the invention for the compound ethyl 3-(acetylbutylamino)propionate, which is known as an insect repellent, to be excluded from the compounds of the formula I.

It has been found that compounds in which R¹ and R³ may be identical or different and are selected from ethyl, n-propyl, isopropyl, n-butyl, isobutyl, tert-butyl, n-pentyl, n-hexyl, n-heptyl, n-octyl, 2-ethylhexyl, n-decyl, n-dodecyl, n-tetradecyl, n-hexadecyl, n-octadecyl, n-eicosyl, n-docosyl and n-tetracosyl, where R¹ preferably stands for 2-ethylhexyl and/or R³ preferably stands for n-octyl, 2-ethylhexyl, n-decyl or n-dodecyl, are particularly suitable for the use according to the invention.

Preference is also given in accordance with the invention to the use of compounds in which R² is selected from the group comprising the elements methyl, n-propyl, isopropyl, n-pentyl, n-heptyl, 1-ethylpentyl, n-nonyl, n-undecyl, where R² is preferably selected from the group comprising the elements methyl, 1-ethyl-pentyl, n-nonyl and n-undecyl.

Specifically, the compounds listed below have proven particularly suitable for the use according to the invention:
ethyl N-acetyl-N-(2-ethylhexyl)-3-aminopropanoate,
ethyl N-acetyl-N-(dodecyl)-3-aminopropanoate,
ethyl N-(2-ethylhexoyl)-N-(butyl)-3-aminopropanoate,
butyl N-acetyl-N-(butyl)-3-aminopropanoate,
ethyl N-(2-ethylhexoyl)-N-(2-ethylhexyl)-3-aminopropanoate,
ethyl N-(2-ethylhexoyl)-N-(dodecyl)-3-aminopropanoate,
butyl N-acetyl-N-(2-ethylhexyl)-3-aminopropanoate,
butyl N-acetyl-N-(dodecyl)-3-aminopropanoate,
butyl N-(2-ethylhexoyl)-N-(butyl)-3-aminopropanoate,
butyl N-(2-ethylhexoyl-N-(2-ethylhexyl)-3-aminopropanoate,
butyl N-(2-ethylhexoyl)-N-(dodecyl)-3-aminopropanoate,
2-ethylhexyl N-acetyl-N-(butyl)-3-aminopropanoate,
2-ethylhexyl N-acetyl-N-(2-ethylhexyl)-3-aminopropanoate,
2-ethylhexyl N-acetyl-N-(dodecyl)-3-aminopropanoate,
2-ethylhexyl N-(2-ethylhexoyl)-N-(butyl)-3-aminopropanoate,
2-ethylhexyl N-(2-ethylhexoyl)-N-(2-ethylhexyl)-3-aminopropanoate,
2-ethylhexyl N-(2-ethylhexoyl)-N-dodecyl)-3-aminopropanoate, where the compound of the formula I is particularly preferably ethyl N-acetyl-N-(2-ethylhexyl)-3-aminopropanoate.

The present application furthermore relates to the novel compounds having the suitability according to the invention. Compounds of the formula I 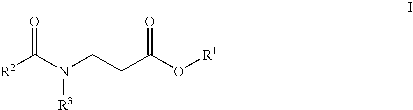
where R¹, R² and R³ may be identical or different and are selected from
straight-chain or branched C₁- to C₂₄-alkyl groups,
straight-chain or branched C₃- to C₂₄-alkenyl groups,
straight-chain or branched C₁- to C₂₄-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
with the proviso that either R¹ stands for 2-ethylhexyl
or R² stands for 1-ethylpentyl and R³ stands for n-octyl, 2-ethylhexyl, n-decyl or n-dodecyl
or R² stands for methyl and R¹ and R³ are different
or the compound of the formula I is butyl N-(2-ethylhexoyl)-N-(butyl)-3-aminopropanoate, are therefore claimed.

Preferred novel compounds are characterised in that R¹ preferably stands for 2-ethylhexyl and/or R³ preferably stands for n-octyl, 2-ethylhexyl, n-decyl or n-dodecyl.

Particularly preferred novel compounds are listed below:
ethyl N-(2-ethylhexoyl)-N-(2-ethylhexyl)-3-aminopropanoate,
ethyl N-(2-ethylhexoyl)-N-(dodecyl)-3-aminopropanoate,
butyl N-acetyl-N-(2-ethylhexyl)-3-aminopropanoate,
butyl N-acetyl-N-(dodecyl)-3-aminopropanoate,
butyl N-(2-ethylhexoyl)-N-(butyl)-3-aminopropanoate,
butyl N-(2-ethylhexoyl-N-(2-ethylhexyl)-3-aminopropanoate,
butyl N-(2-ethylhexoyl)-N-(dodecyl)-3-aminopropanoate,
2-ethylhexyl N-acetyl-N-(butyl)-3-aminopropanoate,
2-ethylhexyl N-acetyl-N-(2-ethylhexyl)-3-aminopropanoate,
2-ethylhexyl N-acetyl-N-(dodecyl)-3-aminopropanoate,
2-ethylhexyl N-(2-ethylhexoyl)-N-(butyl)-3-aminopropanoate,
2-ethylhexyl N-(2-ethylhexoyl)-N-(2-ethylhexyl)-3-aminopropanoate,
2-ethylhexyl N-(2-ethylhexoyl)-N-dodecyl)-3-aminopropanoate.

The compounds which are suitable for the use according to the invention can be prepared by methods known per se: in the preparation, a compound of the formula II 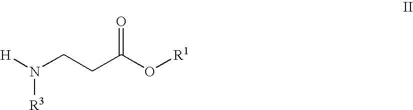
is preferably reacted with an acid derivative R²—C(═O)—X, where X stands for —Cl, —O—C(═O)—R⁴ or —OR⁴, where —R⁴ stands for a straight-chain or branched C₁- to C₂₄-alkyl group, which is preferably identical with R².

In a particularly preferred variant of the preparation process, the acid derivative is an acid anhydride R²—C(═O)—O—C(═O)—R².

The reaction here can be carried out without a solvent or in conventional aprotic solvents. Preferred solvents here may be an ethereal solvent, such as, for example, diethyl ether, tetrahydrofuran (THF), tert-butyl methyl ether (MTBE) or dibutyl ether. However, other polar solvents, such as methyl ethyl ketone and the like, can likewise be used, but also apolar solvents, such as, for example, n-hexane, heptane, toluene, etc.

The compound of the formula II is preferably prepared by reaction of a compound of the formula III 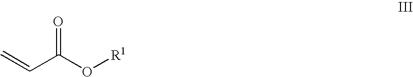
with an amine R³—NH₂. Reaction conditions for these reactions are also familiar to the person skilled in the art and can be adapted without difficulty to the respective radicals R¹ and R³.

The reaction here can be carried out without a solvent or in conventional aprotic solvents. Preferred solvents here may be an ethereal solvent, such as, for example, diethyl ether, tetrahydrofuran (THF), tert-butyl methyl ether (MTBE) or dibutyl ether. However, other polar solvents can likewise be used, but also apolar solvents, such as, for example, n-hexane, heptane, toluene, etc.

The present invention furthermore relates to this preferred preparation process for the novel compounds of the formula I.

The present invention furthermore relates to compositions comprising at least one formulation assistant of the formula I 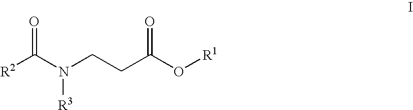
where R¹, R² and R³ may be identical or different and are selected from
straight-chain or branched C₁- to C₂₄-alkyl groups,
straight-chain or branched C₃- to C₂₄-alkenyl groups,
straight-chain or branched C₁- to C₂₄-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
   and at least one active substance whose processing and/or use is simplified by the formulation assistant, where ethyl 3-(acetylbutylamino)-propionate is excluded from the compounds of the formula I.

The compositions are usually compositions which can be used topically, for example cosmetic or dermatological formulations. In this case, the compositions comprise a cosmetically or dermatologically suitable vehicle and, depending on the desired property profile, optionally further suitable ingredients.

The compounds of the formula I are typically employed in accordance with the invention in amounts of 0.01 to 20% by weight, preferably in amounts of 0.1% by weight to 10% by weight and particularly preferably in amounts of 1 to 8% by weight. It causes the person skilled in the art absolutely no difficulties here to select the amounts correspondingly depending on the intended action of the composition.

The active substances to be employed in accordance with the invention include so-called repellents, in particular insect repellents.

A multiplicity of mosquitoes, horseflies, fleas, lice, bugs and ticks and mites, summarised below under the collective terms insects and spiders—or for simplification even under the generic term insects, which is used incorrectly in the biological sense—feed on the blood of mammals, which also include humans. They bore their way into the skin of their victims with their piercing and sucking tools until they hit blood vessels. During feeding, they secrete vessel-dilating and anticoagulant agents which can result in itching, hive formation and allergic reactions in the host. In particular in the tropics and subtropics, there is in addition a risk of infection with pathogens. Thus, for example, malaria is transmitted by the Anopheles mosquito or yellow fever is transmitted by the yellow fever mosquito. In temperate regions too, there is a risk of infections with pathogens transmitted by insects, such as, for example, tick-borne encephalitis transmitted by tick bite.

Protection against annoyance by insects and spiders is offered by repellents. These are taken to mean a number of active substances which have a repellent effect on insects and spiders due to their odour. They are generally low-volatility compounds which evaporate slowly on the skin and thus form a scent pall above the skin which drives away the insects.

The commonest repellents include N,N-diethyl-3-methylbenzamide (“DEET”), which is active against mosquitoes, stable and sand flies, horseflies, fleas, bugs, ticks and mites. Furthermore, dimethyl phthalate (trade name: Palatinol M, DMP) is employed against mosquitoes, lice, ticks and mites. Ethyl 3-(N-n-butyl-N-acetylamino)propionate (available from Merck under the trade name IR3535®), which can be employed, for example, against mosquitoes, tsetse flies and horseflies, is particularly effective.

Most repellent active ingredients belong to the substance classes of the amides, alcohols, esters and ethers. Repellent active ingredients are usually intended to meet the following conditions: they must not evaporate too quickly and must not penetrate into the skin. They must have neither a primarily irritant nor sensitising action on the skin and should in addition be non-toxic. Their efficacy must also be retained on exposure to skin fluid and/or UV radiation.

Accordingly, the compounds of the formula I employed in accordance with the invention as formulation assistants may have a supporting action here in that they
extend the duration of action of the repellents on the skin,
stabilise the repellents against UV radiation and/or skin fluids and/or
enhance the action of the repellents and thus enable the use of smaller amounts of repellent in the formulation.

It is therefore preferred for the composition according to the invention to comprise at least one repellent, where the repellent is preferably selected from N,N-diethyl-3-methylbenzamide, ethyl 3-(acetylbutylamino)propionate, dimethyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, N-(2-ethylhexyl)-8,9,10-trinorborn-5-ene-2,3-dicarboximide, dimethyl carbate, di-n-propyl isocinchomeronate, (R)-p-mentha-1,8-diol, 2-ethylhexane-1,3-diol, N-octylbicycloheptenedicarboximide, piperonyl butoxide, 1-(2-methylpropyloxycarbonyl)-2-(hydroxyethyl)piperidine (Bayrepel®; Bayer) or mixtures thereof, it particularly preferably being selected from N,N-diethyl-3-methylbenzamide, ethyl 3-(acetylbutylamino)-propionate, 1-(2-methylpropyloxycarbonyl)-2-(hydroxyethyl)piperidine or mixtures thereof.

The compositions according to the invention comprising repellents are preferably insect repellents. Insect repellents are available in the form of solutions, gels, sticks, rollers, pump sprays and aerosol sprays, with solutions and sprays forming the majority of the commercially available products. The basis for these two product forms are usually alcoholic or aqueous/alcoholic solutions with addition of fatting substances and slight perfuming. Although other composition forms, such as, in particular, emulsions, creams, ointments and the like, are in principle conceivable and desired, they have, however, to date in some cases proven difficult to formulate in a stable manner. Here too, the formulation assistants according to the invention can advantageously be employed.

In a further preferred embodiment of the present invention, which may also be combined with the embodiment just described, the at least one active substance is an insoluble or sparingly soluble active substance.

It has been found that the use of the compounds of the formula I can result here in an advantageous improvement in the dispersion properties of active substances which are insoluble or sparingly soluble per se.

Insoluble or sparingly soluble active substances of this type are preferably selected here from the group of the organic UV filters, flavone derivatives, chromone derivatives, aryl oximes or parabens.

Parabens are 4-hydroxybenzoic acid esters, which are used in free form or as sodium salts for the preservation of compositions in the area of foods, cosmetics and medicaments. The action of the esters is directly proportional to the chain length of the alkyl radical, but conversely the solubility drops with increasing chain length. As non-dissociating compounds, the esters are substantially pH-independent and act in a pH range from 3.0-8.0. The antimicrobial action mechanism is based on damage of the microbe membranes by the surface activity of the PHB esters and on protein denaturing. In addition, interactions occur with coenzymes. The action is directed against fungi, yeasts and bacteria. The most important parabens as preservatives are methyl 4-hydroxybenzoate, ethyl 4-hydroxybenzoate, propyl 4-hydroxybenzoate, butyl 4-hydroxybenzoate. These compounds, often called only methyl-, ethyl-, propyl- and butylparaben, are only sparingly soluble in common vehicles for the compositions to be preserved. The use of the compounds of the formula I can therefore simplify the incorporation of parabens, in particular methyl 4-hydroxybenzoate, ethyl 4-hydroxybenzoate, propyl 4-hydroxybenzoate, butyl 4-hydroxybenzoate, into compositions.

Of the aryl oximes, which are likewise only sparingly soluble in conventional vehicles for cosmetic and dermatological compositions, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and skin appendages. Compositions according to the invention which, in addition to the compound of the formula I, additionally comprise an aryl oxime, preferably 2-hydroxy-5-methyllaurophenone oxime, exhibit surprising anti-inflammatory suitability. The compositions here preferably comprise 0.01 to 10% by weight of the aryl oxime, it being particularly preferred for the composition to comprise 0.05 to 5% by weight of aryl oxime.

In accordance with the invention, flavone derivatives are taken to mean flavonoids and coumaranones. In accordance with the invention, flavonoids are taken to mean the glycosides of flavonones, flavones, 3-hydroxy-flavones (=flavonols), aurones, isoflavones and rotenoids [Römpp Chemie Lexikon [Römpp's Lexicon of Chemistry], Volume 9, 1993]. For the purposes of the present invention, however, this term is also taken to mean the aglycones, i.e. the sugar-free constituents, and the derivatives of the flavonoids and aglycones. For the purposes of the present invention, the term flavonoid is furthermore also taken to mean anthocyanidine (cyanidine). For the purposes of the present invention, the term coumaranones is also taken to mean derivatives thereof.

Preferred flavonoids are derived from flavonones, flavones, 3-hydroxy-flavones, aurones and isoflavones, in particular from flavonones, flavones, 3-hydroxyflavones and aurones.

The flavonoids are preferably selected from the following compounds: 4,6,3′,4′-tetrahydroxyaurone, quercetin, rutin, isoquercetin, eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin), α-glycosylrutin, tiliroside and the sulfates and phosphates thereof. Of the flavonoids, particular preference is given, as active substances according to the invention, to rutin, tiliroside, α-glycosylrutin and troxerutin.

Of the coumaranones, preference is given to 4,6,3′,4′-tetrahydroxybenzyl-coumaranone-3.

The term chromone derivatives is preferably taken to mean certain chromen-2-one derivatives which are suitable as active ingredients for the preventative treatment of human skin and human hair against ageing processes and harmful environmental influences. At the same time, they exhibit a low irritation potential for the skin, have a positive effect on water binding in the skin, maintain or increase the elasticity of the skin and thus promote smoothing of the skin. These compounds preferably conform to the formula IV 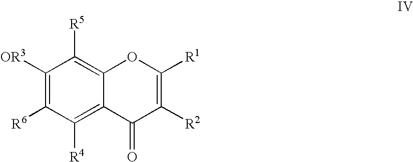
where
R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
R³ stands for H or straight-chain or branched C₁- to C₂₀-alkyl groups,
R⁴ stands for H or OR⁸,
R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen and
R⁷ stands for H, straight-chain or branched C₁- to C₂₀-alkyl groups, a poly-hydroxyl compound, such as preferably an ascorbic acid radical or glycosidic radicals, and
R⁸ stands for H or straight-chain or branched C₁- to C₂₀-alkyl groups, where at least 2 of the substituents R¹, R², R⁴- R⁶ are not H or at least one substituent from R¹ and R² stands for —C(═O)—R⁷ or —C(═O)—OR⁷.

The proportion of one or more compounds selected from flavonoids, chromone derivatives and coumaranones in the composition according to the invention is preferably from 0.001 to 5% by weight, particularly preferably from 0.01 to 2% by weight, based on the composition as a whole.

On use of the dibenzoylmethane derivatives which are particularly preferred as UV-A filters in combination with the compounds of the formula I, an additional advantage arises: the UV-sensitive dibenzoylmethane derivatives are additionally stabilised by the presence of the compounds of the formula I. The present invention therefore furthermore relates to the use of the compounds of the formula I for the stabilisation of dibenzoylmethane derivatives in compositions.

In this case, preferred compositions having light-protection properties comprise at least one dibenzoylmethane derivative. The dibenzoylmethane derivatives used for the purposes of the present invention are, as already shown, products which are already well known per se and are described, in particular, in the above-mentioned documents FR-A-2 326 405, FR-A-2 440 933 and EP-A-0 114 607.

The dibenzoylmethane derivatives which can be used in accordance with the invention may be selected, in particular, from the dibenzoylmethane derivatives of the following formula: 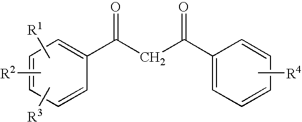
in which R¹, R², R³ and R⁴, which are identical or different from one another, denote hydrogen, a straight-chain or branched C₁₋₈-alkyl group or a straight-chain or branched C₁₋₈-alkoxy group. In accordance with the present invention, it is of course possible to use one dibenzoylmethane derivative or a plurality of dibenzoylmethane derivatives. Of the dibenzoylmethane derivatives to which the present invention specifically relates, mention may be made in particular of:
2-methyldibenzoylmethane,
4-methyldibenzoylmethane,
4-isopropyldibenzoylmethane,
4-tert-butyldibenzoylmethane,
2,4-dimethyidibenzoylmethane,
2,5-dimethyldibenzoylmethane,
4,4′-diisopropyidibenzoylmethane,
4,4′-methoxy-tert-butyldibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane
   and
2,6-dimethyl4-tert-butyl-4′-methoxydibenzoylmethane
   this list being non-restrictive.

Of the above-mentioned dibenzoylmethane derivatives, particular preference is given in accordance with the invention to 4,4′-methoxy-tert-butyl-dibenzoylmethane and especially 4,4′-methoxy-tert-butyldibenzoyl-methane, which is commercially available from Merck under the trade name Eusolexe® 9020, this filter conforming to the following structural formula:

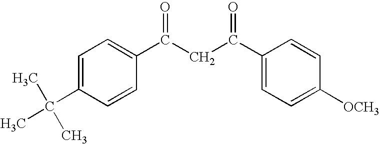

A further dibenzoylmethane derivative which is preferred in accordance with the invention is 4-isopropyidibenzoylmethane.

It has also been observed that compounds of the formula I can have a stabilising action on the composition. On use in corresponding products, these therefore also remain stable for longer and do not change their appearance. In particular, the efficacy of the ingredients, for example vitamins, is retained even on extended application or extended storage. This is, inter alia, particularly advantageous in compositions for protection of the skin against exposure to UV rays, since these cosmetics are subjected to particularly high stresses by the UV radiation.

Compositions which are particularly preferred in accordance with the invention also comprise UV filters in addition to the compounds of the formula I.

In principle, all UV filters are suitable for combination with the compounds of the formula I according to the invention. Particular preference is given to UV filters whose physiological acceptability has already been demonstrated. Both for UVA and UVB filters, there are many proven substances which are known from the specialist literature, for example
benzylidenecamphor derivatives, such as 3-(4′-methylbenzylidene)dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyl-dibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),
phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
   and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]-hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

These organic UV filters are generally incorporated into cosmetic formulations in an amount of 0.5 to 10 per cent by weight, preferably 1-8%.

Further suitable organic UV filters also are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyloxy]poly[oxy(dimethyl)silylene] [and about 6% of methyl[2-[p-[2,2-bis(ethoxycarbonyl]vinyl]phenoxy]-1-methyleneethyl] and about 1.5% of methyl[3-[p-[2,2-bis(ethoxycarbonyl)vinyl)phenoxy)propenyl) and 0.1 to 0.4% of (methylhydrogen)-silylene]] (n≈60) (CAS No. 207 574-74-1)
2,2′-methylenebis(6-(2H-benzotriazol-2-yl)4-(1,1,3,3-tetramethylbutyl)-phenol) (CAS No. 103 59745-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 59745-,187 393-00-6).

Further suitable UV filters are methoxyflavones corresponding to the earlier German patent application DE 10232595.2.

Organic UV filters are generally incorporated into cosmetic formulations in an amount of 0.5 to 20 per cent by weight, preferably 1-15%.

In particular on use of the insoluble or sparingly soluble UV filters from the above list or of organic particulate UV filters, as described, for example, in the patent application WO 99/66896, better distribution of the UV filters in the formulation and thus also more uniform distribution of the UV filters on the skin can be achieved through the use of the formulation assistants according to the invention. The stability, in particular the storage stability, of the formulations, in particular of emulsions, which comprise these UV filters can also be increased by the use of the formulation assistants according to the invention.

In order to ensure optimised UV protection, it is furthermore preferred for compositions having light-protection properties also to comprise inorganic UV filters. Conceivable inorganic UV filters are those from the group of the titanium dioxides, such as, for example, coated titanium dioxide (for example Eusolex® T-2000, Eusolex® T-AQUA), zinc oxides (for example Sachtotec®), iron oxides or also cerium oxides. These inorganic UV filters are generally incorporated into cosmetic compositions in an amount of 0.5 to 20 per cent by weight, preferably 2-10%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzo-phenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

The protective action against damaging effects of UV radiation can be optimised by combining one or more compounds of the formula I with further UV filters.

Optimised compositions may comprise, for example, the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broad-band protection, which can also be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin are repeatedly discussed in the literature. The encapsulation of the corresponding substances which is proposed here suppresses this effect.
In general, encapsulation of individual UV filters or other ingredients enables preparation problems caused by the interaction of individual composition constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00171084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in compositions according to the invention are preferably present in amounts which ensure that the encapsulated UV filters are present in the composition in the above-indicated amounts.

The protective action of compositions according to the invention against oxidative stress or against the action of free radicals can thus be improved further if the compositions comprise one or more antioxidants, where it causes the person skilled in the art absolutely no difficulties to select antioxidants which act suitably quickly or in a delayed manner.

In a preferred embodiment of the present invention, the composition is therefore a composition for the protection of body cells against oxidative stress, in particular for reducing skin ageing, characterised in that it comprises one or more antioxidants besides the one or more compounds of the formula I.

There are many proven substances known from the specialist literature which can be used as antioxidants, for example amino acids (for example glycine, histidine, tyrosine, tryptophan) and derivatives thereof, imidazoles, (for example urocanic acid) and derivatives thereof, peptides, such as D, L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and hepta-thionine sulfoximine) in very low tolerated doses (for example pmol to pmol/kg), and also (metal) chelating agents, (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynexe® L LIQUID), DL-α-tocopherol, L-(+)-ascorbyl palmitate, citric acid and lecithin (for example Oxynexe® LM) or butylhydroxytoluene (BHT), L-(+)-ascorbyl palmitate and citric acid (for example Oxynex® 2004). Antioxidants of this type are usually employed with compounds of the formula I in such compositions in ratios in the range from 1000:1 to 1:1000, preferably in amounts of 100:1 to 1:100.

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. Vitamins are usually employed here with compounds of the formula I in ratios in the range from 1000:1 to 1:1000, preferably in amounts of 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I.M.C.M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A.E.M.F. Soffers, I.M.C.M. Rietjens; Free Radical Biology & Medicine 2001, 31(7), 869-881, are investigating the pH dependence of the antioxidant action of hydroxyflavones. Quercetin exhibits the greatest activity amongst the structures investigated over the entire pH range.

Suitable antioxidants are furthermore compounds of the formula V 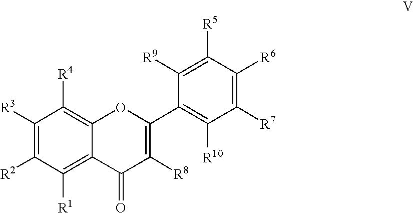
where R¹ to R¹⁰ may be identical or different and are selected from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
where all OR¹¹, independently of one another, stand for
   OH
   straight-chain or branched C₁- to C₂₀-alkoxy groups,
   straight-chain or branched C₃- to C₂₀-alkenyloxy groups,
   straight-chain or branched C₁- to C₂₀-hydroxyalkoxy groups, where the hydroxyl group(s) may be bonded to a primary or secondary carbon atom of the chain and furthermore the alkyl chain may also be interrupted by oxygen, and/or
   C₃- to C₁₀-cycloalkoxy groups and/or C₃- to C₁₂-cycloalkenyloxy groups, where the rings may in each case also be bridged by —(CH₂)ₙ— groups, where n=1 to 3, and/or
   mono- and/or oligoglycosyl radicals,
with the proviso that at least 4 radicals from R¹ to R⁷ stand for OH and that at least 2 pairs of adjacent —OH groups are present in the molecule,
or R², R⁵ and R⁶ stand for OH and the radicals R¹, R³, R⁴ and R⁷⁻¹⁰ stand for H,
   as described in the earlier German patent application DE 10244282.7.

Advantages of the compositions according to the invention comprising at least one antioxidant, besides the above-mentioned advantages, are, in particular, the antioxidant action and the good tolerance by the skin. In addition, preferred compounds of those described here are colourless or only weakly coloured and thus do not result in discoloration of the compositions, or only do so to a small extent. Particularly advantageous is the particular action profile of the compounds of the formula V, which is evident in the DPPH assay from a high capacity for scavenging free radicals (EC₅₀), a delayed action (T_{EC50}>120 min) and thus moderate to high antifree-radical efficiency (AE). In addition, the compounds of the formula V combine in the molecule antioxidative properties with UV absorption in the UV-A and/or -B region. Preference is therefore also given to compositions comprising at least one compound of the formula V which is characterised in that at least two adjacent radicals of the radicals R¹ to R⁴ stand for OH and at least two adjacent radicals of the radicals R⁵ to R⁷ stand for OH. Particularly preferred compositions comprise at least one compound of the formula V which is characterised in that at least three adjacent radicals of the radicals R¹ to R⁴ stand for OH, where the radicals R¹ to R³ preferably stand for OH.

The compositions according to the invention may in addition comprise further conventional skin-protecting or skin-care active ingredients. These may in principle be all active ingredients known to the person skilled in the art.

Particularly preferred active ingredients are, for example, also so-called compatible solutes. These are substances which are involved in the osmoregulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and the respective precursors thereof. For the purposes of German patent application DE-A-10133202, osmolytes are taken to mean, in particular, substances from the group of the polyols, such as, for example, myo-inositol, mannitol or sorbitol and/or one or more of the osmolytically active substances mentioned below:
taurine, choline, betaine, phosphorylcholine, glycerophosphorylcholines, glutamine, glycine, α-alanine, glutamate, aspartate, proline, and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

In accordance with the invention, compatible solutes are preferably substances selected from the group consisting of pyrimidinecarboxylic acids (such as ectoine and hydroxyectoine), proline, betaine, glutamine, cyclic diphosphoglycerate, N-acetylornithine, trimethylamine N-oxide, di-myoinositol phosphate (DIP), cyclic 2,3-diphosphoglycerate (cDPG), 1,1-diglycerol phosphate (DGP), β-mannosyl glycerate (firoin), β-mannosyl-glyceramide (firoin A) or/und dimannosyl diinositol phosphate (DMIP) or an optical isomer, derivative, for example an acid, or a salt or ester of these compounds, or combinations thereof.

Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutical active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula VI 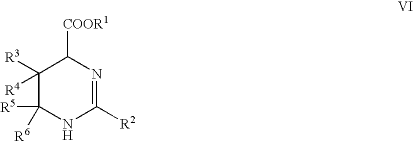
in which R¹ is a radical H or C1-8-alkyl, R² is a radical H or C1-4-alkyl, and R³, R⁴, R⁵ and R⁶ are each, independently of one another, a radical from the group H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). The compositions according to the invention preferably comprise pyrimidinecarboxylic acids of this type in amounts of up to 15% by weight. The pyrimidinecarboxylic acids are preferably employed here in ratios of 100:1 to 1:100 with respect to the compounds of the formula I, with ratios in the range from 1:10 to 10:1 being particularly preferred.

It is particularly preferred in accordance with the invention for the compatible solutes to be selected from di-myo-inositol phosphate (DIP), cyclic 2,3-diphosphoglycerate (cDPG), 1,1-diglycerol phosphate (DGP), β-mannosyl glycerate (firoin), βmannosylglyceramide (firoin-A) or/and di-mannosyl diinositol phosphate (DMIP), ectoine, hydroxyectoine or mixtures thereof.

All compounds or components which can be used in the compositions are either known and commercially available or can be synthesised by known processes.

The one or more compounds of the formula I can be incorporated into cosmetic or dermatological compositions in the customary manner. Suitable compositions are those for external use, for example in the form of a cream, lotion, gel, or as a solution which can be sprayed onto the skin. Suitable for internal use are administration forms such as capsules, coated tablets, powders, tablet solutions or solutions.

Examples which may be mentioned of application forms of the compositions according to the invention are: solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower products. Any desired customary vehicles, assistants and, if desired, further active ingredients may be added to the composition.

Preferred assistants originate from the group of the preservatives, antioxidants, stabilisers, solubilisers, vitamins, colorants, odour enhancers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, mascara, eyeliner, eyeshadow, rouge, powder make-up, emulsion make-up and wax make-up, and sunscreen, pre-sun and after-sun preparations.

The preferred composition forms according to the invention include, in particular, emulsions.

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of esters of this type, for example jojoba oil.

The oil phase may furthermore advantageously be selected from the group of the branched and unbranched hydrocarbons and waxes, silicone oils, dialkyl ethers, the group of the saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24 C atoms, in particular 12-18 C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyidodecanol, isotridecyl isononanoate, isoeicosane, 2-ethylhexyl cocoate, C₁₂₋₁₅-alkyl benzoate, caprylic/capric acid triglyceride, dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the compositions according to the invention comprise hydrophilic surfactants.

The hydrophilic surfactants are preferably selected from the group of the alkylglucosides, acyl lactylates, betaines and coconut amphoacetates.

The alkylglucosides are themselves advantageously selected from the group of the alkylglucosides which are distinguished by the structural formula 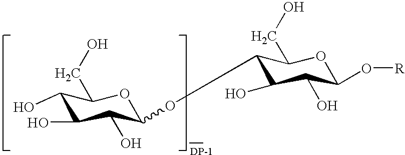
where R represents a branched or unbranched alkyl radical having 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = Σ \frac{{p}_{i}}{100} \cdot i$$

in which p₁, p₂, p₃ . . . pᵢ represent the proportion of mono-, di-, tri- . . . i-fold glucosylated products in per cent by weight. Products having degrees of glucosylation of 1-2, particularly advantageously of 1.1 to 1.5, very particularly advantageously of 1.2-1.4, in particular of 1.3, are advantageously selected in accordance with the invention.

The value DP takes into account the fact that alkylglucosides generally, as a consequence of their preparation, represent mixtures of mono- and oligoglucosides. A relatively high content of monoglucosides, typically in the order of 40-70% by weight, is advantageous in accordance with the invention.

Alkylglucosides which are particularly advantageously used in accordance with the invention are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active ingredients used in accordance with the invention, for example Plantarene® 1200 (Henkel KGaA), Oramixe® NS 10 (Seppic).

The acyllactylates are themselves advantageously selected from the group of the substances which are distinguished by the structural formula 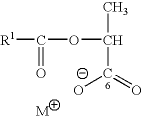
where R¹ denotes a branched or unbranched alkyl radical having 1 to 30 carbon atoms, and M⁺ is selected from the group of the alkali metal ions and the group of the ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group of the substances which are distinguished by the structural formula 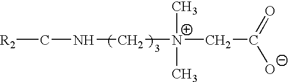
where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageous in accordance with the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied to the skin and/or the hair in an adequate amount in the usual manner for cosmetics.

Cosmetic and dermatological compositions according to the invention may exist in various forms. Thus, they may be, for example, a solution, a waterfree composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageous in accordance with the invention are, for example, O/W emulsifiers, principally from the group of the substances having HLB values of 11-16, very particularly advantageously having HLB values of 14.5-15.5, so long as the O/W emulsifiers have saturated radicals R and R′. If the ONV emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:
polyethylene glycol (20) stearate, polyethylene glycol (21) stearate, polyethyiene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group c polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:
fatty alcohols having 8 to 30 C atoms, monoglycerol esters of saturated and/or unsaturated, branched and/or unbranched alkanecarboxylic acids having a chain length of 8 to 24 C atoms, in particular 12-18 C atoms, diglycerol esters of saturated and/or unsaturated, branched and/or unbranched alkanecarboxylic acids having a chain length of from 8 to 24 C atoms, in particular 12-18 C atoms, monoglycerol ethers of saturated and/or unsaturated, branched and/or unbranched alcohols having a chain length of 8 to 24 C atoms, in particular 12-18 C atoms, diglycerol ethers of saturated and/or unsaturated, branched and/or unbranched alcohols having a chain length of 8 to 24 C atoms, in particular 12-18 C atoms, propylene glycol esters of saturated and/or unsaturated, branched and/or unbranched alkanecarboxylic acids having a chain length of 8 to 24 C atoms, in particular 12-18 C atoms, and sorbitan esters of saturated and/or unsaturated, branched and/or unbranched alkanecarboxylic acids having a chain length of 8 to 24 C atoms, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Preferred compositions in accordance with the invention are particularly suitable for protecting human skin against ageing processes and against oxidative stress, i.e. against damage caused by free radicals, as are produced, for example, by solar irradiation, heat or other influences. In this connection, they are in the various administration forms usually used for this application. For example, they may, in particular, be in the form of a lotion or emulsion, such as in the form of a cream or milk (O/W, W/O, O/W/O, W/O/W), in the form of oily/alcoholic, oily/aqueous or aqueous/alcoholic gels or solutions, in the form of solid sticks or may be formulated as an aerosol.

The composition may comprise cosmetic adjuvants which are usually used in this type of composition, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substances, a lower monoalcohol or lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which, apart from the compound(s) of the formula 1, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily/alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily/alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The cosmetic composition may also be used to protect the hair against photochemical damage in order to prevent colour changes, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling and treating the hair, in the form of a lotion or gel for brushing or blow-waving, in the form of a hair lacquer, permanent-wave composition, colorant or bleach for the hair. Besides the compound(s) of the formula I, the composition having light-protection properties may comprise various adjuvants used in this type of composition, such as surface-active agents, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The present invention furthermore relates to a process for the preparation of a composition which is characterised in that at least one compound of the formula I containing radicals as described above is mixed with a vehicle which is suitable cosmetically or dermatologically or for foods, and to the use of a compound of the formula I for the preparation of a composition having antioxidant properties.

The compositions according to the invention can be prepared using techniques which are well known to the person skilled in the art.

The mixing can result in dissolution, emulsification or dispersion of the compound of the formula I in the vehicle.

The invention is explained in greater detail below by means of examples. The invention can be carried out throughout the range claimed and is not restricted to the examples given here.

### EXAMPLES

### Example 1

### Preparation of Butyl N-acetyl-N-(2-ethylhexyl)-3-aminopropionate

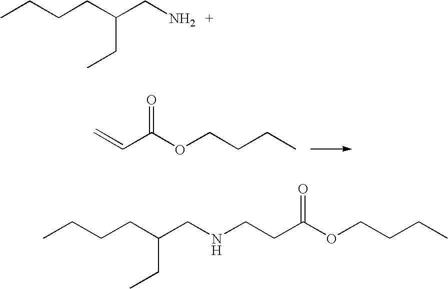

0.78 mol (100.2 g) of 2-ethylhexylamine are initially introduced under nitrogen in a 1 l four-necked flask and cooled to ˜0° C. in an ice bath. 1.56 mol (202.1 g) of ethyl acrylate are added dropwise with stirring over the course of about 30 minutes, during which the internal temperature does not rise above 5° C. The clear, colourless reaction mixture is stirred for a further one hour. The reaction mixture is distilled under reduced pressure in order to separate off unreacted ethyl acrylate and 2-ethylhexylamine. Yield: 191.6 g (88% content-corrected) of product (GC content ˜93.0%)

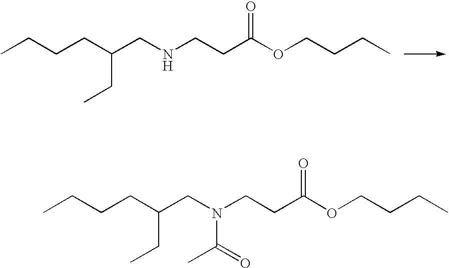

0.69 mol (191 g) of butyl 3-(2-ethylhexyl)aminopropanoate are initially introduced under nitrogen in a 500 ml four-necked flask and cooled to ˜0° C. in an ice bath. 0.92 mol (93.9 g) of acetic anhydride are added dropwise over the course of about 20 minutes. The reaction mixture warms to ˜22° C. by the end of the addition. In order to complete the reaction, the reaction mixture is heated to ˜120° C. in an oil bath and stirred at this temperature for about a further 1 hour. The reaction solution is cooled, and 0.22 mol (49) of water is added at ˜75° C. in order to hydrolyse the excess acetic anhydride. The mixture is stirred for a further hour with slow cooling. The acetic acid formed is subsequently distilled off under reduced pressure. For further purification, the distillation residue is chromatographed over 900 g of neutral aluminium oxide using tert-butyl methyl ether. The product-containing fractions are combined and concentrated to dryness. The product is subsequently degassed in a high vacuum. Yield: 148.0 g (72% content-corrected) of product (GC content ˜99.9%).

The following compounds are prepared analogously from the correspondingly modified starting materials by the principle described:
ethyl N-(2-ethylhexoyl)-N-(2-ethylhexyl3-aminopropanoate,
ethyl N-(2-ethylhexoyl)-N-(dodecyl)-3-aminopropanoate,
butyl N-acetyl-N-(dodecyl)-3-aminopropanoate,
butyl N-(2-ethylhexoyl)-N-(butyl)-3-aminopropanoate,
butyl N-(2-ethylhexoyl-N-(2-ethylhexyl)-3-aminopropanoate,
butyl N-(2-ethylhexoyl)-N-(dodecyl)-3-aminopropanoate,
2-ethylhexyl N-acetyl-N-(butyl)-3-aminopropanoate,
2-ethylhexyl N-acetyl-N-(2-ethylhexyl)-3-aminopropanoate,
2-ethylhexyl N-acetyl-N-(dodecyl)-3-aminopropanoate,
2-ethylhexyl N-(2-ethylhexoyl)-N-(butyl)-3-aminopropanoate,
2-ethylhexyl N-(2-ethylhexoyl)-N-(2-ethylhexyl)-3-aminopropanoate,
2-ethylhexyl N-(2-ethylhexoyl)-N-dodecyl)-3-aminopropanoate.

### Example 2

### Liquid

| Ingredient (INCl) | [%] |
|---|---|
| A | |
| Butyl N-acetyl-N-(2-ethylhexyl )-3-aminopropanoate | 25 |
| POLYSORBATE 80 | 0.20 |
| ACRYLATES/C10-30 ALKYL ACRYLATE | 0.20 |
| CROSSPOLYMER | |
| PARFUM | q.s. |
| B | |
| Ethanol 96% | 20.00 |
| AQUA (WATER) | to 100 |
| PROPYLENE GLYCOL | 10.00 |
| C | |
| SODIUM HYDROXIDE | q.s. |

Preparation:

Phases A and B are mixed separately, and phase A is added to phase B with stirring. The pH is adjusted by means of phase C, and the mixture is homogenised.

### Example 3

### O/W After-Sun Lotion

| Ingredient (INCl) | [%] |
|---|---|
| A | |
| Ethyl N-(2-ethylhexoyl)-N-(2-ethylhexyl)-3-aminopropanoate | 10.00 |
| BISABOLOL | 0.30 |
| CETEARYL ALCOHOL, CETEARYL | 4.00 |
| GLUCOSIDE | |
| CAPRYLIC/CAPRIC TRIGLYCERIDE | 2.00 |
| CYCLOPENTASILOXANE | 2.00 |
| DIMETHICONE | 1.00 |
| B | |
| AQUA (WATER) | to 100 |
| GLYCERIN | 3.00 |
| Preservatives | q.s. |
| CITRIC ACID | 0.07 |
| DISODIUM PHOSPHATE | 0.59 |
| C | |
| XANTHAN GUM | 0.50 |

Preparation:

Phases A and B are heated separately to 75° C., phase C is slowly added to B, and the mixture is homogenised. A is subsequently added to B/C at 75° C., and the mixture is homogenised.

### Example 4

### Aqueous Gel

| Ingredient (INCl) | [%] |
|---|---|
| A | |
| PROPYLENE GLYCOL | 5.00 |
| ALLANTOIN | 0.20 |
| AQUA (WATER) | to 100 |
| B | |
| CARBOMER | 33.30 |
| C | |
| Triethanolamine | 3.00 |
| D | |
| Ethyl N-(2-ethylhexoyl)-N-(2-ethylhexyl)-3-aminopropanoate | 10.00 |

Preparation:

Phase A is heated to 75° C. and slowly added to B. C is subsequently added to A/B, and the mixture is homogenised. D is then added.

### Example 5

### Spray

| Ingredient (INCl) | [%] |
|---|---|
| A | |
| Ethyl N-(2-ethylhexoyl)-N-(dodecyl)-3-aminopropanoate | 20.00 |
| PEG-8 | 5.00 |
| PPG-15 STEARYL ETHER | 3.00 |
| PARFUM | 0.30 |
| B | |
| ALCOHOL | 35.00 |
| PEG-32 | 4.00 |
| POLYSORBATE 20 | 1.50 |
| AQUA (WATER) | to 100 |

Preparation:

Phase B is slowly added to A, and the mixture is homogenised.

### Example 6

### Roll-On

| Ingredient (INCl) | [%] |
|---|---|
| A | |
| 2-Ethylhexyl N-acetyl-N-(butyl)-3-aminopropanoate | 20.00 |
| PEG-8 | 5.00 |
| PPG-15 STEARYL ETHER | 3.00 |
| PARFUM | 0.30 |
| B | |
| ALCOHOL | 35.00 |
| PEG-32 | 4.00 |
| POLYSORBATE 20 | 1.50 |
| CARBOMER | 10.00 |
| AQUA (WATER) | to 100 |
| C | |
| TRIETHANOLAMINE | 0.30 |

Preparation:

Phases A and B are mixed separately, and phase B is added to phase A with stirring. The pH is adjusted by means of phase C, and the mixture is subsequently homogenised.

### Example 7

### Cream

| Ingredient (INCl) | [%] |
|---|---|
| A | |
| 2-Ethylhexyl N-acetyl-N-(butyl)-3-aminopropanoate | 20.00 |
| CETEARYL ALCOHOL, CETEARYL GLUCOSIDE | 4.00 |
| CARBOMER | 0.25 |
| B | |
| Preservatives | q.s. |
| GLYCERIN | 3.00 |
| XANTHAN GUM | 0.50 |
| AQUA (WATER) | to 100 |
| C | |
| TRIETHANOLAMINE | q.s. |

Preparation:

Phases A and B are heated to 70° C., and A is slowly added to B. The pH is adjusted by means of phase C, and the mixture is subsequently homogenised.

### Example 8

### Cream

| Ingredient (INCl) | [%] |
|---|---|
| A | |
| 2-Ethylhexyl N-acetyl-N-(butyl)-3-aminopropanoate | 20.00 |
| CETEARYL ALCOHOL, CETEARYL GLUCOSIDE | 4.00 |
| CARBOMER | 0.40 |
| OLEYL ERUCATE | 1.00 |
| CYCLOPENTASILOXANE | 1.00 |
| DIBUTYL ADIPATE | 1.00 |
| ISOPROPYL PALMITATE | 1.00 |
| B | |
| GLYCERIN | 3.00 |
| XANTHAN GUM | 0.20 |
| Preservatives | q.s. |
| AQUA (WATER) | to 100 |
| C | |
| TRIETHANOLAMINE | q.s. |

Preparation:

Phases A and B are heated to 70° C., and A is slowly added to B. The pH is adjusted by means of phase C, and the mixture is subsequently homogenised.

### Example 9

### Camouflage Paint

| Ingredient (INCl) | [%] |
|---|---|
| A | |
| Cl 77499 (IRON OXIDES), MICA | 15.00 |
| TALC | 15.00 |
| B | |
| C18-36 ACID TRIGLYCERIDE | 12.00 |
| TRIBEHENIN | 3.00 |
| CAPRYLIC/CAPRIC TRIGLYCERIDE | 7.00 |
| STEARIC ACID | 3.00 |
| PEG-8, TOCOPHEROL, ASCORBYL | 0.10 |
| PALMITATE, ASCORBIC ACID, CITRIC ACID | |
| Preservatives | q.s. |
| PPG-2 MYRISTYL ETHER PROPIONATE | to 100 |
| 2-Ethylhexyl N-acetyl-N-(butyl)-3-aminopropanoate | 20.00 |

Preparation:

Phase A is mixed and slowly added to phase B heated to 80° C.

### Example 10

### Sunscreen (SPF: 32)

| Ingredient (INCl) | [%] |
|---|---|
| A | |
| 2-Ethylhexyl N-(2-ethylhexoyl)-N-(butyl)-3-aminopropanoate | 7.50 |
| ETHYLHEXYL METHOXYCINNAMATE, BHT | 7.50 |
| OCTOCRYLENE | 10.00 |
| BUTYL METHOXYDIBENZOYLMETHANE | 0.90 |
| BENZOPHENONE-3 | 1.10 |
| GLYCERYL STEARATE, PEG-100 STEARATE | 3.20 |
| COCO-GLUCOSIDE, COCONUT ALCOHOL | 1.30 |
| DIBUTYL ADIPATE | 5.50 |
| TOCOPHERYL ACETATE | 0.25 |
| B | |
| LAURETH-7, POLYACRYLAMIDE, C13-14-ISOPARAFFIN | 1.20 |
| C | |
| GLYCERIN | 7.00 |
| XANTHAN GUM | 0.15 |
| MAGNESIUM ALUMINUM SILICATE | 0.60 |
| DISODIUM EDTA | 0.10 |
| AQUA (WATER) | to 100 |
| D | |
| Preservatives | q.s. |
| PARFUM | 0.20 |
| E | |
| TROMETHAMINE | q.s. |

Preparation:

All phases are prepared separately. Phase C: Xanthan Gum and Magnesium Aluminium Silicate are heated to 75-80° C. and mixed with the other constituents. Phase A is added to phase C at 75° C. After homogenisation, phase B is added at 60° C. After homogenisation, phase D is added at 40° C. Phase E is then added (pH=6.5).

### Example 11

### Sunscreen (SPF: 43.6)

| Ingredient (INCl) | [%] |
|---|---|
| A | |
| 2-Ethylhexyl N-(2-ethylhexoyl)-N-(butyl)-3-aminopropanoate | 10.00 |
| TOCOPHERYL ACETATE | 0.25 |
| ETHYLHEXYL METHOXYCINNAMATE, BHT | 7.50 |
| OCTOCRYLENE | 10.00 |
| BUTYL METHOXYDIBENZOYLMETHANE | 0.90 |
| BENZOPHENONE-3 | 1.10 |
| GLYCERYL STEARATE, PEG-100 STEARATE | 3.20 |
| COCO-GLUCOSIDE, COCONUT ALCOHOL | 1.30 |
| DIBUTYL ADIPATE | 3.00 |
| B | |
| LAURETH-7, POLYACRYLAMIDE, C13-14-ISOPARAFFIN | 1.20 |
| Cyclopentasiloxane | 5.00 |
| C | |
| GLYCERIN | 7.00 |
| XANTHAN GUM | 0.15 |
| MAGNESIUM ALUMINUM SILICATE | 0.60 |
| DISODIUM EDTA | 0.10 |
| AQUA (WATER) | to 100 |
| D | |
| Preservatives | q.s. |
| PARFUM | 0.20 |
| E | |
| TROMETHAMINE | q.s. |

Preparation:

All phases are prepared separately. Phase C: Xanthan Gum and Magnesium Aluminium Silicate are heated to 75-80° C. and mixed with the other constituents. Phase A is added to phase C at 75° C. After homogenisation, phase B is added at 60° C. After homogenisation, phase D is added at 40° C. Phase E is then added (pH=6.5).

### Example 12

### Sunscreen (SPF: 28.3)

| Ingredient (INCl) | [%] |
|---|---|
| A | |
| PROPYLENE GLYCOL | 2.00 |
| AQUA (WATER) | to 100 |
| CARBOMER | 0.30 |
| B | |
| 2-ethylhexyl N-(2-ethylhexyl)-N-(butyl)-3-aminopropanoate | 7.50 |
| GLYCERYL STEARATE, PEG-100 STEARATE | 2.00 |
| BENZOPHENONE-3 | 5.00 |
| ETHYLHEXYL SALICYLATE | 5.00 |
| ETHYLHEXYL METHOXYCINNAMATE, BHT | 10.00 |
| 4-METHYLBENZYLIDENE CAMPHOR | 2.00 |
| ISOPROPYL PALMITATE | 1.00 |
| CETYL PEG/PPG-10/1 DIMETHICONE | 2.00 |
| CYCLOPENTASILOXANE | 1.00 |
| TRIMETHYLSILOXYSILICATE, DIMETHICONE | 1.00 |
| C | |
| Preservatives | q.s. |
| PARFUM | q.s. |
| TRIETHANOLAMINE | 0.30 |

Preparation:

Phases A and B are heated to 75° C., and B is slowly added to A. Phase C is added at 40° C., and the mixture is subsequently homogenised.

### Example 13

### Sunscreen (SPF: 28.3)

| Ingredient (INCl) | [%] |
|---|---|
| A | |
| PROPYLENE GLYCOL | 2.00 |
| AQUA (WATER) | to 100 |
| CARBOMER | 0.30 |
| B | |
| 2-Ethylhexyl N-(2-ethylhexyl)-N-(acetyl)-3-aminopropanoate | 10.00 |
| GLYCERYL STEARATE, PEG-100 STEARATE | 2.00 |
| BENZOPHENONE-3 | 5.00 |
| ETHYLHEXYL SALICYLATE | 5.00 |
| ETHYLHEXYL METHOXYCINNAMATE, BHT | 10.00 |
| 4-METHYLBENZYLIDENE CAMPHOR | 2.00 |
| ISOPROPYL PALMITATE | 1.00 |
| CETYL PEG/PPG-10/1 DIMETHICONE | 2.00 |
| CYCLOPENTASILOXANE | 1.00 |
| TRIMETHYLSILOXYSILICATE, DIMETHICONE | 1.00 |
| C | |
| Preservatives | q.s. |
| PARFUM | q.s. |
| TRIETHANOLAMINE | 0.30 |

Preparation:

Phases A and B are heated to 75° C., and B is slowly added to A. Phase C is added at 40° C., and the mixture is subsequently homogenised.

### Example 14

### Spray

| Ingredient (INCl) | [%] |
|---|---|
| A | |
| 2-Ethylhexyl N-(2-ethylhexyl)-N-(acetyl)-3-aminopropanoate | 6.25 |
| BENZOPHENONE-3 | 1.50 |
| ETHYLHEXYL METHOXYCINNAMATE, BHT | 3.50 |
| DICAPRYLYL CARBONATE | 0.50 |
| DECYL OLEATE | 0.25 |
| POLYGLYCERYL-3 METHYLGLUCOSE DISTEARATE | 1.00 |
| ISOPROPYL PALMITATE | 0.50 |
| B | |
| AQUA (WATER) | to 100 |
| GLYCERIN | 3.00 |
| Preservatives | q.s. |
| CARBOMER | 0.10 |
| SODIUM HYDROXIDE | 0.25 |
| C | |
| PARFUM | 0.20 |

Preparation:

Phases A and B are heated to 80° C., and A is slowly added to B. Phase C is added at 40° C., and the mixture is subsequently homogenised.

### Example 15

### Spray

| Ingredient (INCl) | [%] |
|---|---|
| A | |
| 2-Ethylhexyl N-(2-ethylhexyl)-N-(2-ethylhexoyl)-3- | 7.50 |
| aminopropanoate | |
| BENZOPHENONE-3 | 1.50 |
| ETHYLHEXYL METHOXYCINNAMATE, BHT | 3.50 |
| DICAPRYLYL CARBONATE | 0.50 |
| DECYL OLEATE | 0.25 |
| POLYGLYCERYL-3 METHYLGLUCOSE DISTEARATE | 1.00 |
| ISOPROPYL PALMITATE | 0.50 |
| DIETHYLHEXYL CARBONATE | 0.50 |
| B | |
| AQUA (WATER) | to 100 |
| GLYCERIN | 3.00 |
| Preservatives | q.s. |
| CARBOMER | 0.10 |
| SODIUM HYDROXIDE | 0.25 |
| C | |
| PARFUM | 0.20 |

Preparation:

Phases A and B are heated to 80° C., and A is slowly added to B. Phase C is added at 40° C., and the mixture is subsequently homogenised.

### Example 16

### Spray

| Ingredient (INCl) | [%] |
|---|---|
| A | |
| Ethyl N-(2-ethylhexoyl)-N-(2-ethylhexyl)-3-aminopropanoate | 25.00 |
| STEARETH-21 | 2.50 |
| Arlatone 985 | 5.00 |
| PARAFFINUM LIQUIDUM (MINERAL OIL) | 5.00 |
| DIMETHICONE | 1.00 |
| MYRISTYL ALCOHOL | 4.00 |
| B | |
| AQUA (WATER) | to 100 |
| GLYCERIN | 3.00 |
| CARBOMER | 1.00 |
| C | |
| Preservatives | q.s. |
| D | |
| TROMETHAMINE | 0.10 |
| AQUA (WATER) | 1.00 |

Preparation:

Phase A is heated to 75° C. and slowly added to B. C is subsequently added to A/B, and the mixture is homogenised. D is then added in order to set the pH.

### Example 17

### Spray

| Ingredient (INCl) | [%] |
|---|---|
| A | |
| Ethyl N-(2-ethylhexoyl)-N-(2-ethylhexyl)-3-aminopropanoate | 22.50 |
| STEARETH-21 | 2.00 |
| Arlatone 985 | 4.00 |
| PARAFFINUM LIQUIDUM (MINERAL OIL) | 7.50 |
| DIMETHICONE | 1.00 |
| MYRISTYL ALCOHOL | 4.00 |
| B | |
| AQUA (WATER) | to 100 |
| GLYCERIN | 3.00 |
| CARBOMER | 0.10 |
| C | |
| Preservatives | q.s. |
| D | |
| TROMETHAMINE | 0.15 |
| AQUA (WATER) | 1.50 |

Preparation:

Phase A is heated to 75° C. and slowly added to B. C is subsequently added to A/B, and the mixture is homogenised. D is then added in order to set the pH.

### Example 18

### Emulsion

| Ingredient (INCl) | [%] |
|---|---|
| A | |
| Ethyl N-(2-ethylhexoyl)-N-(dodecyl)-3-aminopropanoate | 25.00 |
| MYRISTYL ALCOHOL, MYRISTYL GLUCOSIDE | 5.00 |
| ISOSTEARYL ISOSTEARATE | 2.00 |
| CETYL ALCOHOL | 2.00 |
| B | |
| AQUA (WATER) | to 100 |
| CARBOMER | 0.40 |
| C | |
| SODIUM HYDROXIDE | q.s. |
| D | |
| Preservatives | q.s. |
| PARFUM | q.s. |

Preparation:

Phase A is heated to 80° C. and slowly added to B (75° C.). C is subsequently added to A/B, and the mixture is homogenised. D is then added.

### Example 19

### O/W After-Sun Lotion

| Ingredient (INCl) | [%] |
|---|---|
| A | |
| Ethyl N-(2-ethylhexoyl)-N-(2-ethylhexyl)-3-aminopropanoate | 10.00 |
| BISABOLOL | 0.30 |
| CETEARYL ALCOHOL, CETEARYL | 4.00 |
| GLUCOSIDE | |
| CAPRYLIC/CAPRIC TRIGLYCERIDE | 2.00 |
| CYCLOPENTASILOXANE | 2.00 |
| DIMETHICONE | 1.00 |
| B | |
| AQUA (WATER) | to 100 |
| GLYCERIN | 3.00 |
| Preservatives | q.s. |
| C | |
| XANTHAN GUM | 0.50 |

Preparation:

Phases A and B are heated separately to 75° C., phase C is slowly added to B, and the mixture is homogenised. A is subsequently added to B/C at 75° C., and the mixture is homogenised.

### Example 20

### Compositions

Formulations for cosmetic compositions comprising compounds of the formula I are indicated below by way of example. The INCI names of the commercially available compounds are also indicated.

UV Pearl, OMC stands for the composition having the INCI name:
Water (for EU: Aqua), Ethylhexyl Methoxycinnamate, Silica, PVP, chlorophenesin, BHT; this composition is commercially available from Merck KGaA, Darmstadt, under the name Eusolex®UV Pearl™OMC.

The other UV Pearl indicated in the tables each have an analogous composition, with OMC being replaced by the UV filters indicated.

TABLE 1
W/O emulsions (data in % by weight)

| | 1-1 | 1-2 | 1-3 | 1-4 | 1-5 | 1-6 | 1-7 | 1-8 | 1-9 | 1-10 |
|---|---|---|---|---|---|---|---|---|---|---|
| Titanium Dioxide | | 2 | 5 | | | | | | | 3 |
| Ethyl N-(2-Ethylhexoyl)-N-(2- | 5 | 3 | 2 | 1 | 2 | | | | 1 | 1 |
| ethylhexyl)-3-amino- | | | | | | | | | | |
| propanoate | | | | | | | | | | |
| Ethyl N-(2-Ethylhexoyl)-N- | | | | | | 1 | 2 | 1 | | |
| (acetyl)-3-aminopropanoate | | | | | | | | | | |
| Zinc Oxide | | | | | | | | 5 | 2 | |
| UV-Pearl, OMC | 30 | 15 | 15 | 15 | 15 | 15 | 15 | 15 | 15 | 15 |
| Polyglyceryl 3-Dimerate | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Water | to | to | to | to | to | to | to | to | to | to |
| | 100 | 100 | 100 | 100 | 100 | 100 | 100 | 100 | 100 | 100 |

| | 1-11 | 1-12 | 1-13 | 1-14 | 1-15 | 1-16 | 1-17 | 1-18 |
|---|---|---|---|---|---|---|---|---|
| Titanium Dioxide | 3 | | 2 | | 3 | | 2 | 5 |
| Benzylidene Malonate Polysiloxane | | 1 | 0.5 | | | | | |
| Methylene Bis-Benzotriazolyl | 1 | 1 | 0.5 | | | | | |
| Tetramethylbutylphenol | | | | | | | | |
| Ethyl N-(2-Ethylhexoyl)-N-(2-ethyl- | 5 | 3 | 2 | 5 | 1 | 3 | 7 | 2 |
| hexyl)-3-aminopropanoate | | | | | | | | |
| Polyglyceryl 3-Dimerate | 3 | 3 | 3 | 3 | | | | |
| Cera Alba | 0.3 | 0.3 | 0.3 | 0.3 | 2 | 2 | 2 | 2 |
| Hydrogenated Castor Oil | 0.2 | 0.2 | 0.2 | 0.2 | | | | |
| Paraffinium Liquidum | 7 | 7 | 7 | 7 | | | | |
| Caprylic/Capric Triglyceride | 7 | 7 | 7 | 7 | | | | |
| Hexyl Laurate | 4 | 4 | 4 | 4 | | | | |
| PVP/Eicosene Copolymer | 2 | 2 | 2 | 2 | | | | |
| Propylene Glycol | 4 | 4 | 4 | 4 | | | | |
| Magnesium Sulfate | 0.6 | 0.6 | 0.6 | 0.6 | | | | |
| Tocopherol | 0.5 | 0.5 | 0.5 | 0.5 | | | | |
| Tocopheryl Acetate | 0.5 | 0.5 | 0.5 | 0.5 | 1 | 1 | 1 | 1 |
| Cyclomethicone | 0.5 | 0.5 | 0.5 | 0.5 | | | | |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Dicocoyl Pentaerythrityl Citrate | | | | | 6 | 6 | 6 | 6 |
| (and) Sorbitan Sesquioleate (and) Cera | | | | | | | | |
| Alba (and) Aluminium Stearate | | | | | | | | |
| PEG-7 Hydrogenated Castor Oil | | | | | 1 | 1 | 1 | 1 |
| Zinc Stearate | | | | | 2 | 2 | 2 | 2 |
| Oleyl Erucate | | | | | 6 | 6 | 6 | 6 |
| Decyl Oleate | | | | | 6 | 6 | 6 | 6 |
| Dimethicone | | | | | 5 | 5 | 5 | 5 |
| Tromethamine | | | | | 1 | 1 | 1 | 1 |
| Glycerin | | | | | 5 | 5 | 5 | 5 |
| Allantoin | | | | | 0.2 | 0.2 | 0.2 | 0.2 |
| Water | to | to | to | to | to | to | to | to |
| | 100 | 100 | 100 | 100 | 100 | 100 | 100 | 100 |

| | 1-19 | 1-20 | 1-21 | 1-22 | 1-23 | 1-24 | 1-25 | 1-26 | 1-27 | 1-28 | 1-29 |
|---|---|---|---|---|---|---|---|---|---|---|---|
| Titanium Dioxide | | 2 | 5 | | | | | | | 3 | 3 |
| Benzylidene Malonate Polysiloxane | | | | 1 | | | | | 1 | 1 | |
| Zinc Oxide | | | | | | | | 5 | 2 | | |
| Ethyl N-(2-Ethylhexoyl)-N-(2-ethyl- | 5 | 5 | 5 | 5 | 7 | 5 | 5 | 5 | 5 | 5 | 8 |
| hexyl)-3-aminopropanoate | | | | | | | | | | | |
| UV-Pearl, OCR | | 10 | | | | | | | | | 5 |
| UV-Pearl, Ethylhexyl Dimethyl PABA | | | 10 | | | | | | | | |
| UV-Pearl, Homosalate | | | | 10 | | | | | | | |
| UV-Pearl, Ethylhexyl salicylate | | | | | 10 | | | | | | |
| UV-Pearl, OMC, BP-3 | | | | | | 10 | | | | | |
| UV-Pearl, OCR, BP-3 | | | | | | | 10 | | | | |
| UV-Pearl, Ethylhexyl Dimethyl PABA, | | | | | | | | 10 | | | |
| BP-3 | | | | | | | | | | | |
| UV-Pearl, Homosalate, BP-3 | | | | | | | | | 10 | | |
| UV-Pearl, Ethylhexyl Salicylate, BP-3 | | | | | | | | | | 10 | |
| BMDBM | | | | | | | | | | | 2 |
| UV-Pearl OMC, 4-Methylbenzylidene | 25 | | | | | | | | | | |
| Camphor | | | | | | | | | | | |
| Polyceryl 3-Dimerate | 3 | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 | 0.15 |

| | |
|---|---|
| Water | to 100 |

TABLE 2
O/W emulsions, data in % by weight

| | 2-1 | 2-2 | 2-3 | 2-4 | 2-5 | 2-6 | 2-7 | 2-8 | 2-9 | 2-10 |
|---|---|---|---|---|---|---|---|---|---|---|
| Titanium Dioxide | | 2 | 5 | | | | | | | 3 |
| Methylene Bis-Benzotriazolyl | | | | | | 1 | 2 | 1 | | |
| Tetramethylbutylphenol | | | | | | | | | | |
| 2-(1-Ethylhexyl)-5,7-dihydroxy- | | | | 1 | 2 | | | | 1 | 1 |
| chromen-4-one | | | | | | | | | | |
| 4′-Methoxy-6-hydroxyflavone | 1 | 3 | | 2 | | 5 | | 5 | 2 | |
| Ethyl N-(Acetyl)-N-(2-ethylhexyl)-3- | 5 | 5 | 5 | 5 | 5 | 5 | 5 | 5 | 5 | 5 |
| aminopropanoate | | | | | | | | | | |
| 2-Carboxyl-5,7-dihydroxy-chromen- | 1 | 5 | 4 | | 6 | | 7 | | 2 | 1 |
| 4-one | | | | | | | | | | |
| 4-Methylbenzylidene Camphor | 2 | | 3 | | 4 | | 3 | | 2 | |
| BMDBM | 1 | 3 | | 3 | 3 | | 3 | 3 | 3 | |
| Stearyl Alcohol (and) Steareth-7 | 3 | 3 | 3 | 3 | 3 | 3 | 3 | 3 | 3 | 3 |
| (and) Steareth-10 | | | | | | | | | | |
| Glyceryl Stearate (and) Ceteth-20 | 3 | 3 | 3 | 3 | 3 | 3 | 3 | 3 | 3 | 3 |
| Glyceryl Stearate | 3 | 3 | 3 | 3 | 3 | 3 | 3 | 3 | 3 | 3 |
| Microwax | 1 | 1 | 1 | 1 | 1 | 1 | 1 | 1 | 1 | 1 |
| Cetearyl Octanoate | 11.5 | 11.5 | 11.5 | 11.5 | 11.5 | 11.5 | 11.5 | 11.5 | 11.5 | 11.5 |
| Caprylic/Capric Triglyceride | 6 | 6 | 6 | 6 | 6 | 6 | 6 | 6 | 6 | 6 |
| Oleyl Oleate | 6 | 6 | 6 | 6 | 6 | 6 | 6 | 6 | 6 | 6 |
| Propylene Glycol | 4 | 4 | 4 | 4 | 4 | 4 | 4 | 4 | 4 | 4 |
| Glyceryl Stearate SE | | | | | | | | | | |
| Stearic Acid | | | | | | | | | | |
| Persea Gratissima | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Glycerin | | | | | | | | | | |
| Water | to | to | to | to | to | to | to | to | to | to |
| | 100 | 100 | 100 | 100 | 100 | 100 | 100 | 100 | 100 | 100 |

| | 2-11 | 2-12 | 2-13 | 2-14 | 2-15 | 2-16 | 2-17 | 2-18 |
|---|---|---|---|---|---|---|---|---|
| Titanium Dioxide | 3 | | 2 | | | | 2 | 5 |
| Benzylidene Malonate Polysiloxane | | 1 | 0.5 | | | | | |
| Methylene Bis-Benzotriazolyl | 1 | 1 | 0.5 | | | | | |
| Tetramethylbutylphenol | | | | | | | | |
| 4′-Methoxy-7-β-glucosidylflavone | | | | 1 | 2 | | | |
| 2-Carboxyl-5,7-dihydroxychromen-4- | 1 | 3 | | 2 | | 5 | | 5 |
| one | | | | | | | | |
| Ethyl N-(Acetyl)-N-(2-ethylhexyl)-3- | 5 | 5 | 5 | 5 | 5 | 5 | 5 | 5 |
| aminopropanoate | | | | | | | | |
| Ethyl 5,7-dihydroxychromen-4-one- | 1 | 5 | 4 | | 6 | | 7 | |
| 2-carboxylate | | | | | | | | |
| Zinc Oxide | | | 2 | | | | | |
| UV-Pearl, OMC | 15 | 15 | 15 | 30 | 30 | 30 | 15 | 15 |
| 4-Methylbenzylidene Camphor | | | | 3 | | | | |
| BMDBM | | | | 1 | | | | |
| Phenylbenzimidazole Sulfonic Acid | | | | | 4 | | | |
| Stearyl Alcohol (and) Steareth-7 | 3 | 3 | 3 | 3 | | | | |
| (and) Steareth-10 | | | | | | | | |
| Glyceryl Stearate (and) Ceteth-20 | 3 | 3 | 3 | 3 | | | | |
| Glyceryl Stearate | 3 | 3 | 3 | 3 | | | | |
| Microwax | 1 | 1 | 1 | 1 | | | | |
| Cetearyl Octanoate | 11.5 | 11.5 | 11.5 | 11.5 | | | | |
| Caprylic/Capric Triglyceride | 6 | 6 | 6 | 6 | 14 | 14 | 14 | 14 |
| Oleyl Oleate | 6 | 6 | 6 | 6 | | | | |
| Propylene Glycol | 4 | 4 | 4 | 4 | | | | |
| Glyceryl Stearate SE | | | | | 6 | 6 | 6 | 6 |
| Stearic Acid | | | | | 2 | 2 | 2 | 2 |
| Persea Gratissima | | | | | 8 | 8 | 8 | 8 |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Tromethamine | | | | | 1.8 | | | |
| Glycerin | | | | | 3 | 3 | 3 | 3 |
| Water | to | to | to | to | to | to | to | to |
| | 100 | 100 | 100 | 100 | 100 | 100 | 100 | 100 |

| | 2-19 | 2-20 | 2-21 | 2-22 | 2-23 | 2-24 | 2-25 | 2-26 | 2-27 | 2-28 |
|---|---|---|---|---|---|---|---|---|---|---|
| Titanium Dioxide | | | | | | | 3 | 3 | | 2 |
| Benzylidene Malonate | 1 | 2 | | | | 1 | 1 | | 1 | 0.5 |
| Polysiloxane | | | | | | | | | | |
| 7,8,3′,4′-Tetrahydroxyflavone | | | | 1 | 2 | | | | 1 | 1 |
| Ethyl 5,7-Dihydroxychromen-4- | 1 | 3 | | 2 | | 5 | | 5 | 2 | |
| one-2-carboxylate | | | | | | | | | | |
| Ethyl N-(Acetyl)-N-(dodecyl)-3- | 5 | 5 | 5 | 5 | 5 | 5 | 5 | 5 | 5 | 5 |
| aminopropanoate | | | | | | | | | | |
| Methylene Bis-Benzotriazolyl | | | 1 | 2 | 1 | | | 1 | 1 | 0.5 |
| Tetramethylbutylphenol | | | | | | | | | | |
| Zinc Oxide | | | | | 5 | 2 | | | | 2 |
| UV-Pearl, OMC | 15 | 15 | 15 | 15 | 15 | 15 | 15 | 15 | 15 | 15 |
| Caprylic/Capric Triglyceride | 14 | 14 | 14 | 14 | 14 | 14 | 14 | 14 | 14 | 14 |
| Oleyl Oleate | | | | | | | | | | |
| Propylene Glycol | | | | | | | | | | |
| Glyceryl Stearate SE | 6 | 6 | 6 | 6 | 6 | 6 | 6 | 6 | 6 | 6 |
| Stearic Acid | 2 | 2 | 2 | 2 | 2 | 2 | 2 | 2 | 2 | 2 |
| Persea Gratissima | 8 | 8 | 8 | 8 | 8 | 8 | 8 | 8 | 8 | 8 |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Glycerin | 3 | 3 | 3 | 3 | 3 | 3 | 3 | 3 | 3 | 3 |
| Water | to | to | to | to | to | to | to | to | to | to |
| | 100 | 100 | 100 | 100 | 100 | 100 | 100 | 100 | 100 | 100 |

TABLE 3
Gels, data in % by weight

| | 3-1 | 3-2 | 3-3 | 3-4 | 3-5 | 3-6 | 3-7 | 3-8 | 3-9 | 3-10 |
|---|---|---|---|---|---|---|---|---|---|---|
| a = aqueous gel | | | | | | | | | | |
| Titanium Dioxide | | 2 | 5 | | | | | | | 3 |
| 2-Methyl-5,7-dihydroxychromen-4- | | | | 1 | 2 | | | | 1 | 1 |
| one | | | | | | | | | | |
| Ethyl 5,7-Dihydroxychromen-4-one- | 1 | 3 | | 2 | | 5 | | 5 | 2 | |
| 2-carboxylate | | | | | | | | | | |
| Benzylidene Malonate Polysiloxane | | | 1 | 1 | 2 | | | | 1 | 1 |
| Methylene Bis-Benzotriazolyl | | 1 | | | | 1 | 2 | 1 | | |
| Tetramethylbutylphenol | | | | | | | | | | |
| Zinc Oxide | | | | 2 | | | | 5 | 2 | |
| Ethyl N-(Butyl)-N-(2-ethylhexyl)-3- | 30 | 15 | 15 | 15 | 15 | 15 | 15 | 15 | 15 | 15 |
| aminopropanoate | | | | | | | | | | |
| 4-Methylbenzylidene Camphor | | | | | 2 | | | | | |
| Butylmethoxydibenzoylmethane | | 1 | | | | | | | | |
| Phenylbenzimidazole Sulfonic Acid | | | | 4 | | | | | | |
| Prunus Dulcis | 5 | 5 | 5 | 5 | 5 | 5 | 5 | 5 | 5 | 5 |
| Tocopheryl Acetate | 0.5 | 0.5 | 0.5 | 0.5 | 0.5 | 0.5 | 0.5 | 0.5 | 0.5 | 0.5 |
| Caprylic/Capric Triglyceride | 3 | 3 | 3 | 3 | 3 | 3 | 3 | 3 | 3 | 3 |
| Octyldodecanol | 2 | 2 | 2 | 2 | 2 | 2 | 2 | 2 | 2 | 2 |
| Decyl Oleate | 2 | 2 | 2 | 2 | 2 | 2 | 2 | 2 | 2 | 2 |
| PEG-8 (and) Tocopherol (and) | 0.05 | 0.05 | 0.05 | 0.05 | 0.05 | 0.05 | 0.05 | 0.05 | 0.05 | 0.05 |
| Ascorbyl Palmitate (and) Ascorbic | | | | | | | | | | |
| Acid (and) Citric Acid | | | | | | | | | | |
| Sorbitol | 4 | 4 | 4 | 4 | 4 | 4 | 4 | 4 | 4 | 4 |
| Polyacrylamide (and) C13-14 | 3 | 3 | 3 | 3 | 3 | 3 | 3 | 3 | 3 | 3 |
| Isoparaffin (and) Laureth-7 | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Water | to | to | to | to | to | to | to | to | to | to |
| | 100 | 100 | 100 | 100 | 100 | 100 | 100 | 100 | 100 | 100 |

1. Use of a compound of the formula I 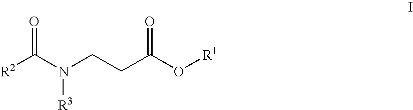
where R¹, R² and R³ may be identical or different and are selected from
straight-chain or branched C₁- to C₂₄-alkyl groups,
straight-chain or branched C₃- to C₂₄-alkenyl groups,
straight-chain or branched C₁- to C₂₄-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
as formulation assistant for the preparation of cosmetic or dermatological compositions.

2. Use according to claim 1 of a compound of the formula I according to claim 1, where the formulation assistant serves as solubiliser.

3. Use according to claim 1 of a compound of the formula I according to claim 1, where the formulation assistant serves as penetration enhancer.

4. Use according to claim 1 of a compound of the formula I according to claim 1, where the formulation assistant serves as action enhancer.

5. Use according to claim 1, characterised in that ethyl 3-(acetylbutylamino)propionate is excluded from the compounds of the formula I.

6. Use according to claim 1, characterised in that R¹ and R³ may be identical or different and are selected from ethyl, n-propyl, isopropyl, n-butyl, iso-butyl, tert-butyl, n-pentyl, n-hexyl, n-heptyl, n-octyl, 2-ethylhexyl, n-decyl, n-dodecyl, n-tetradecyl, n-hexadecyl, n-octadecyl, n-eicosyl, n-docosyl and n-tetracosyl, where R¹ preferably stands for 2-ethylhexyl and/or R³ preferably stands for n-octyl, 2-ethylhexyl, n-decyl or n-dodecyl.

7. Use according to claim 1, characterised in that R² is selected from the group comprising the elements methyl, n-propyl, isopropyl, n-pentyl, n-heptyl, 1-ethylpentyl, n-nonyl, n-undecyl, where R² is preferably selected from the group comprising the elements methyl, 1-ethylpentyl, n-nonyl and n-undecyl.

8. Use according to claim 1, characterised in that the compound of the formula I is selected from the compounds ethyl N-acetyl-N-(2-ethylhexyl)-3-aminopropanoate, ethyl N-acetyl-N-(dodecyl)-3-aminopropanoate, ethyl N-(2-ethylhexoyl)-N-(butyl)-3-aminopropanoate, butyl N-acetyl-N-(butyl)-3-aminopropanoate, ethyl N-(2-ethylhexoyl)-N-(2-ethylhexyl)-3-aminopropanoate, ethyl N-(2-ethylhexoyl)-N-(dodecyl)-3-aminopropanoate, butyl N-acetyl-N-(2-ethylhexyl)-3-aminopropanoate, butyl N-acetyl-N-(dodecyl)-3-aminopropanoate, butyl N-(2-ethylhexoyl)-N-(butyl)-3-aminopropanoate, butyl N-(2-ethylhexoyl-N-(2-ethylhexyl)-3-aminopropanoate, butyl N-(2-ethylhexoyl)-N-(dodecyl)-3-aminopropanoate, 2-ethylhexyl N-acetyl-N-(butyl)-3-aminopropanoate, 2-ethylhexyl N-acetyl-N-(2-ethylhexyl)-3-aminopropanoate, 2-ethylhexyl N-acetyl-N(dodecyl)-3-aminopropanoate, 2-ethylhexyl N-(2-ethylhexoyl)-N-(butyl)-3-aminopropanoate, 2-ethylhexyl N-(2-ethylhexoyl)-N-(2-ethylhexyl)-3-aminopropanoate, 2-ethylhexyl N-(2-ethylhexoyl)-N-dodecyl)-3-aminopropanoate, where the compound of the formula I is particularly preferably ethyl N-acetyl-N-(2-ethylhexyl)-3-aminopropanoate.

9. Compound of the formula I 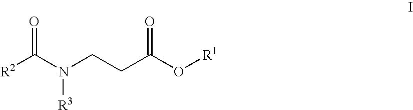
where R¹, R² and R³ may be identical or different and are selected from
straight-chain or branched C₁- to C₂₄-alkyl groups,
straight-chain or branched C₃- to C₂₄-alkenyl groups,
straight-chain or branched C₁- to C₂₄-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
with the proviso that either R¹ stands for 2-ethylhexyl
or R² stands for 1-ethylpentyl and R³ stands for n-octyl, 2-ethylhexyl, n-decyl or n-dodecyl
or R² stands for methyl and R¹ and R³ are different
or the compound of the formula I is butyl N-(2-ethylhexoyl)-N-(butyl)-3-aminopropanoate.

10. Compound according to claim 9, characterised in that R¹ preferably stands for 2-ethylhexyl and/or R³ preferably stands for n-octyl, 2-ethylhexyl, n-decyl or n-dodecyl.

11. Compound according to claim 9, characterised in that the compound of the formula I is selected from the compounds ethyl N-(2-ethylhexoyl)-N-(2-ethylhexyl)-3-aminopropanoate, ethyl N-(2-ethylhexoyl)-N-(dodecyl)-3-aminopropanoate, butyl N-acetyl-N-(2-ethylhexyl)-3-aminopropanoate, butyl N-acetyl-N-(dodecyl)-3-aminopropanoate, butyl N-(2-ethylhexoyl)-N-(butyl)-3-aminopropanoate, butyl N-(2-ethylhexoyl-N-(2-ethylhexyl)-3-aminopropanoate, butyl N-(2-ethylhexoyl)-N-(dodecyl)-3-aminopropanoate, 2-ethylhexyl N-acetyl-N-(butyl)-3-aminopropanoate, 2-ethylhexyl N-acetyl-N-(2-ethylhexyl)-3-aminopropanoate, 2-ethylhexyl N-acetyl-N-(dodecyl)-3-aminopropanoate, 2-ethylhexyl N-(2-ethylhexoyl)-N-(butyl)-3-aminopropanoate, 2-ethylhexyl N-(2-ethylhexoyl)-N-(2-ethylhexyl)-3-aminopropanoate, 2-ethylhexyl N-(2-ethylhexoyl)-N-dodecyl)-3-aminopropanoate.

12. Process for the preparation of a compound of the formula I according to claim 9, characterised in that a compound of the formula II 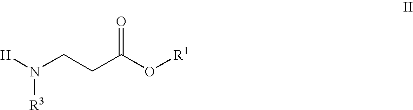
is reacted with an acid derivative R²—C(═O)—X, where X stands for —Cl, —O—C(═O)—R⁴ or —OR⁴, where —R⁴ stands for a straight-chain or branched C₁- to C₂₄-alkyl group, which is preferably identical with R².

13. Process for the preparation of a compound of the formula I according to claim 12, characterised in that the compound of the formula II is obtained by reaction of a compound of the formula III 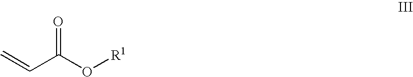
with an amine R³—NH₂.

14. Composition comprising at least one formulation assistant of the formula I 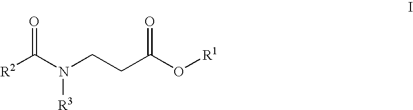
where R¹, R² and R³ may be identical or different and are selected from
straight-chain or branched C₁- to C₂₄-alkyl groups,
straight-chain or branched C₃- to C₂₄-alkenyl groups,
straight-chain or branched C₁- to C₂₄-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may in each case also be bridged by —(CH₂)ₙ— groups, where n=1 to 3,
and at least one active substance whose processing and/or use is simplified by the formulation assistant, where ethyl 3-(acetylbutylamino)-propionate is excluded from the compounds of the formula I.

15. Composition according to claim 14, characterised in that the at least one active substance is at least one repellent, preferably selected from N,N-diethyl-3-methylbenzamide, ethyl 3-(acetylbutylamino)propionate, dimethyl phthalate, butopyronoxyl, 2,3,4,5-bis(2-butylene)-tetrahydro-2-furaldehyde, N,N-diethylcaprylamide, N,N-diethylbenzamide, o-chloro-N,N-diethylbenzamide, dimethyl carbate, di-n-propyl isocinchomeronate, 2-ethylhexane-1,3-diol, N-octylbicycloheptenedicarboximide, piperonyl butoxide, 1-(2-methylpropyloxycarbonyl)-2-(hydroxyethyl)piperidine or mixtures thereof, at least one repellent particularly preferably being selected from N,N-diethyl-3-methylbenzamide, ethyl 3-(acetylbutylamino)propionate, 1-(2-methylpropyloxycarbonyl)-2-(hydroxyethyl)piperidine or mixtures thereof.

16. Composition according to claim 14, characterised in that the at least one active substance is an insoluble or sparingly soluble active substance selected from the group of the organic UV filters, flavone derivatives, chromone derivatives, aryl oximes or parabens.

17. Composition according to claim 1 comprising at least one compound of the formula I, characterised in that the composition comprises one or more compounds of the formula I in an amount of 0.01 to 20% by weight, preferably in an amount of 0.1 to 10% by weight.

18. Composition according to claim 1 for the protection of body cells against oxidative stress, in particular for reducing skin ageing, characterised in that it preferably comprises one or more further antioxidants and/or vitamins, preferably selected from vitamin A palmitate, vitamin C and derivatives thereof, DL-α-tocopherol, tocopherol E acetate, nicotinic acid, pantothenic acid and biotin.

19. Composition according to claim 1, where the composition, besides the at least one compound of the formula I, comprises one or more UV filters, which are preferably selected from the group of 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

20. Process for the preparation of a composition, characterised in that a compound of the formula I containing radicals according to claim 1 is mixed with a cosmetically or dermatologically suitable vehicle.

21. Use of a compound of the formula I, where the variables have the meaning indicated in claim 1, for the preparation of a composition which is suitable for topical application.
